(19) United States
(12) Patent Application Publication
(10) Pub. No.: US 2020/0215018 A1
(43) Pub. Date: Jul. 9, 2020

(54) Gold(I) And Platinum(II) With Isocyanide Ligand Complexes: Synthesis And Biological Activity

(71) Applicant: BOARD OF TRUSTEES OF THE UNIVERSITY OF ARKANSAS, Fayetteville, AR (US)

(72) Inventors: Mohammad Hassan Beyzavi, Fayetteville, AR (US); Hamid Reza Shahsavari, Fayetteville, AR (US)

(73) Assignee: BOARD OF TRUSTEES OF THE UNIVERSITY OF ARKANSAS, Fayetteville, AR (US)

(21) Appl. No.: 16/737,821

(22) Filed: Jan. 8, 2020

Related U.S. Application Data
(60) Provisional application No. 62/789,981, filed on Jan. 8, 2019.

(51) Int. Cl. A61K 31/282; A61K 31/675; A61P 35/00

(52) U.S. Cl. CPC A61K 31/282; A61P 35/00; A61K 31/675

(57) ABSTRACT

A method of treating cancer which comprises administering to a cancer patient a therapeutically effective amount of a gold or platinum based drug that has less toxicity and a different mechanism of action to kill the tumor cells than cisplatin.

### RELATED APPLICATIONS

This application claims priority to U.S. Provisional Application Ser. No. 62/789981 filed on Jan. 8, 2019, which is incorporated herein by reference.

### STATEMENT REGARDING FEDERALLY SPONSORED RESEARCH & DEVELOPMENT

Not applicable.

### INCORPORATION BY REFERENCE OF MATERIAL SUBMITTED ON A COMPACT DISC

Not applicable.

### BACKGROUND OF THE INVENTION

Cisplatin and its analogs, specifically, carboplatin and oxaliplatin, are well-known chemotherapeutic drugs, while nedaplatin, lobaplatin, miriplatin, and heptaplatin are subsequently developed as potential chemotherapeutic agents. These compounds are important clinical chemotherapeutic drugs and they play a vital role in cancer treatment, but these drugs have severe side effects on normal cell lines and produce serious problems like hematological toxicity and neurotoxicity, and also some solid tumors have gained resistance to them.

The disadvantages and limitations of the current drugs have highly encouraged organometallic and biomedical research to concentrate on the discovery of new platinum compounds. In this regard, some platinum complexes have been synthesized and tested on various cancer lines. Most of these compounds were excluded in primary clinical steps of treatment due to their toxicity. Therefore, novel platinum drugs that are less toxic and more selective than currently available drugs against the resistance tumors are in high demand. As a result, there is continuous research to discover new platinum-based drugs with lower toxicity and also to get around the body resistance towards the current commercial drugs.

Because of the development of body resistance towards platinum anticancer drugs, on the other hand, novel gold-based anti-tumor complexes with pharmacological characteristics other than Pt-based drugs are important targets in modern drug design and medicinal chemistry. In recent years, Au-based complexes have received increasing consideration because of their potent inhibition of cancer cell growth which is mainly caused by non-cisplatin-like mechanisms of action.

### BRIEF SUMMARY OF THE INVENTION

In one embodiment, the present invention concerns gold-based drugs that have less toxicity and have a different mechanism of action to kill the tumor cells.

In other embodiments, the present invention concerns gold-based drugs that have less toxicity and have a different mechanism of action to kill the tumor cells as well as having antimicrobial properties.

In one embodiment, the present invention concerns platinum-based drugs that have less toxicity and have a different mechanism of action to kill the tumor cells.

In other embodiments, the present invention concerns platinum-based drugs that have less toxicity and have a different mechanism of action to kill the tumor cells as well as having antimicrobial properties.

In other embodiments, the present invention concerns Pt(II) complexes synthesized by the reaction of the precursor complexes cis,cis-[PtMe₂(μ-SMe₂)₂PtMe₂], and cis-[(p-tolyl)₂Pt(SMe₂)₂], with four and two equivalents of different types of isocyanide ligands (CNR; R=a; t-butyl, b; benzyl, and c; cyclohexyl isocyanide), respectively.

In other embodiments, the present invention concerns the reaction of [(Me₂S)AuCl] with benzyl isocyanide (PhCH₂NC) ligands that led to the formation of a complex [(PhCH₂NC)AuCl] (1).

In other embodiments, the present invention concerns a series of closely related platinum(II) complexes with general formula trans-[Pt(PPh₂allyl)₂(κ¹—S—SR)₂], 3, PPh₂allyl=allyldiphenylphosphine, SR=deprotonated form of pyridine-2-thiol (Spy, 3a), 5-(trifluoromethyl)-pyridine-2-thiol (SpyCF₃-5, 3b), pyrimidine-2-thiol (SpyN, 3c), benzothiazole-2-thiol (Sbt, 3d) and benzimidazole-2-thiol (Sbi, 3e), which were successfully synthesized by reacting a starting complex cis-[Pt(PPh₂allyl)₂Cl₂], A, with corresponding thionate ligands.

In another embodiment, the present invention provides a method of treating cancer which comprises administering to a cancer patient a therapeutically effective amount of one or more metal precursor complexes with one or more ligands.

In another embodiment, the present invention provides a method of treating cancer wherein the metal precursor is cis,cis-[PtMe₂(μ-SMe₂)₂PtMe₂].

In another embodiment, the present invention provides a method of treating cancer wherein the ligand is an isocyanide, the isocyanide ligand is [Me₂Pt(CNR)₂](R=t-butyl).

In another embodiment, the present invention provides a method of treating cancer wherein the ligand is an isocyanide, the isocyanide ligand is [Me₂Pt(CNR)₂] (R=benzyl).

In another embodiment, the present invention provides a method of treating cancer wherein the ligand is an isocyanide, the isocyanide ligand is [Me₂Pt(CNR)₂] (R=cyclohexyl).

In another embodiment, the present invention provides a method of treating cancer wherein the metal precursor is cis-[(p-tolyl)₂Pt(SMe₂)₂].

In another embodiment, the present invention provides a method of treating cancer wherein the ligand is an isocyanide, the isocyanide ligand is [(p-tolyl)₂Pt(CNR)₂] (R=t-butyl).

In another embodiment, the present invention provides a method of treating cancer wherein the ligand is an isocyanide, the isocyanide ligand is [(p-tolyl)₂(CNR)₂] (R=benzyl).

In another embodiment, the present invention provides a method of treating cancer wherein the ligand is an isocyanide, the isocyanide ligand is [(p-tolyl)₂(CNR)₂] (R=cyclohexyl).

In another embodiment, the present invention provides a method of treating cancer wherein the isocyanide ligand is [Me₂Pt(CNR)₂] (R=benzyl) and the complex binds to a DNA minor grove.

In another embodiment, the present invention provides a method of treating cancer wherein methyl groups attach to the metal precursor and are placed away from the base pairs in the DNA minor groove.

In another embodiment, the present invention provides a method of treating cancer wherein the benzyl groups bonded to the isocyanide fit into the minor groove of DNA.

In another embodiment, the present invention provides a method of treating cancer wherein the complex interacts via benzyl CH₂ groups with C9, G10, G4, and C11 base pairs of the DNA.

In another embodiment, the present invention provides a method of treating cancer wherein the metal precursor is [(Me₂S)AuCl] and the ligand, the ligand is a benzyl isocyanide (PhCH₂NC) and forms complex [(PhCH₂NC)AuCl].

In another embodiment, the present invention provides a method of treating cancer wherein the chloride ligand is replaced by pyrimidine-2-thiolate (SpyN⁻) to form complex [(PhCH₂NC) Au(κ¹—S-Spy).

In another embodiment, the present invention provides a method of treating cancer wherein the metal precursor is [Pt(PPh₂allyl)₂Cl₂] and the ligand is a thionate ligand.

In another embodiment, the present invention provides a method of treating cancer which comprises administering to a cancer patient a therapeutically effective amount of platinum complex having a formula trans-[Pt(PPh₂allyl)₂(κ¹—S-SR)₂], PPh₂allyl=allyldiphenylphosphine and SR is a deprotonated form of pyridine-2-thiol.

In another embodiment, the present invention provides a method of treating cancer wherein SR from the group comprising 5-(trifluoromethyl)-pyridine-2-thiol; pyrimidine-2-thiol; benzothiazole-2-thiol; or benzimidazole-2-thiol.

In another embodiment, the present invention provides a method of treating cancer which comprises administering to a cancer patient a therapeutically effective amount of a formula having Pt(II) complexes synthesized by the reaction of the precursor complexes cis,cis-[Me₂Pt(μ-SMe₂)₂PtMe₂], and cis-[(p-tolyl)₂Pt(SMe₂)₂], with four and two equivalents of different types of isocyanide ligands (CNR; R=a; t-butyl, b; benzyl, and c; cyclohexyl isocyanide), respectively.

### BRIEF DESCRIPTION OF THE SEVERAL VIEWS OF THE DRAWINGS

In the drawings, which are not necessarily drawn to scale, like numerals may describe substantially similar components throughout the several views. Like numerals having different letter suffixes may represent different instances of substantially similar components. The drawings illustrate generally, by way of example, but not by way of limitation, a detailed description of certain embodiments discussed in the present document.

FIG. 1 illustrates a synthetic route for the preparation of Pt(II) complexes with isocyanide ligands for an embodiment of the present invention.

FIG. 2 shows the IC₅₀ of the Pt(II) complexes against A549, SKOV3, and MCF-7 cancer cell lines for an embodiment of the present invention.

FIG. 3 shows, for an embodiment of the present invention, flow cytometry-based detection of the apoptotic properties of MCF-7 cells. Representative scatter plots show apoptosis of MCF-7 cells after 48 hours of incubation with different concentrations of (20, 40, and 80 mM) using the Annexin V-PE/7AAD detection kit. The percentages of apoptotic cells (Q2: late apoptotic and Q3: early apoptotic) were determined in Annexin V cells. Q1: necrotic cells, Q2: late apoptotic cells, Q3: early apoptotic cells, and Q4: living cells.

FIG. 4 depicts 2D and 3D ligand-receptor interactions with DNA (PDB ID: 1LU5) for an embodiment of the present invention.

FIG. 5 illustrates an electrophoresis mobility shift assay for platinum compounds for an embodiment of the present invention. A pCDNA plasmid in the circular form was incubated with cisplatin, 1b, and 2a at different concentrations for 24 h (A) and 48 h (B).

FIG. 6 illustrates a synthetic route for the preparation of complexes for additional embodiments of the present invention.

FIG. 7 illustrates a flow cytometric analysis of apoptotic effect of 1a. MCF-7 cells (Human breast carcinoma) were left untreated (A) or treated for 48 h with 2.5, 5 and 10 μM of 1a. Q1: necrotic cells, Q2: late apoptotic cells, Q3: early apoptotic cells, and Q4: living cells for additional embodiments of the present invention.

FIG. 8 shows the effect of 1a on the cell cycle in MCF-7 cells for additional embodiments of the present invention.

FIG. 9 depicts the Genotoxic effect of 1a on MCF-7 cell line. The percentage of degraded DNA in the tail has remarkably increase following treatment with doxorubicin (B: Doxorubicin) as positive control and different concentrations of 1a (C and D) in comparison to untreated cells (A: negative control). For better resolution, the same pictures from CometScore software are also shown for additional embodiments of the present invention.

FIG. 10 is a mobility shift assay of 1a compound. pGEM-FT plasmid in circular form was incubated with different concentrations of cisplatin (positive controls) as well as compound 1a for 24 h for additional embodiments of the present invention.

FIG. 11 shows the generation of ROS in SKOV3 cells induced by various concentrations of 1a and H202 as positive control. Changes in ROS levels were expressed as a ratio of the mean fluorescence intensity (MFI) in each condition divided by the basal intensity of the ROS at the untreated cells (negative controls, MFI0). H₂O₂ could induce ROS production within 20 minutes (line DD). Compound la at all concentrations could induce ROS production in SKOV3 cell line, however in lower concentration, 10 μM (line AA), is more efficient than higher concentrations, 20 μM (line BB) and 40 μM (line CC), in comparison to untreated cells (area under line EE) for additional embodiments of the present invention.

### DETAILED DESCRIPTION OF THE INVENTION

Detailed embodiments of the present invention are disclosed herein; however, it is to be understood that the disclosed embodiments are merely exemplary of the invention, which may be embodied in various forms. Therefore, specific structural and functional details disclosed herein are not to be interpreted as limiting, but merely as a representative basis for teaching one skilled in the art to variously employ the present invention in virtually any appropriately detailed method, structure or system. Further, the terms and phrases used herein are not intended to be limiting, but rather to provide an understandable description of the invention.

The present invention in a first embodiment concerns the synthesis, characterization, and chemotherapeutic properties of platinum(II) complexes containing isocyanide ligands (CNR=t-butyl, benzyl, and cyclohexyl isocyanide). Docked modeling suggests that the complexes bind to DNA via a groove binding mode. Accordingly, the results indicate that the Pt complexes of the present invention have significant anticancer applications.

The present invention in a second embodiment concerns a new sulfur-based gold complex. The linear d¹⁰ two-coordinate gold complex is readily prepared in high yield by replacement of dimethylsulfide ligand with benzyl isocyanide. NMR spectroscopy and X-ray diffraction confirmed the structure of the gold complex. The chemistry of the gold complex was explored in a salt metathesis reaction. In this reaction, an anion exchange between the gold complex precursor and potassium pyrimidine-2-thiolate occurs leading to the formation of a gold complex product. The cytotoxic activities of gold complex precursor and gold complex product 2 were screened against various cancer cell lines. The in vitro results revealed that the gold complex product had reasonable IC50 and the highest activity, while a gold complex precursor displayed less cytotoxic activity. Additional mechanistic investigation revealed that gold complex product induced significant cancer cell death by apoptosis.

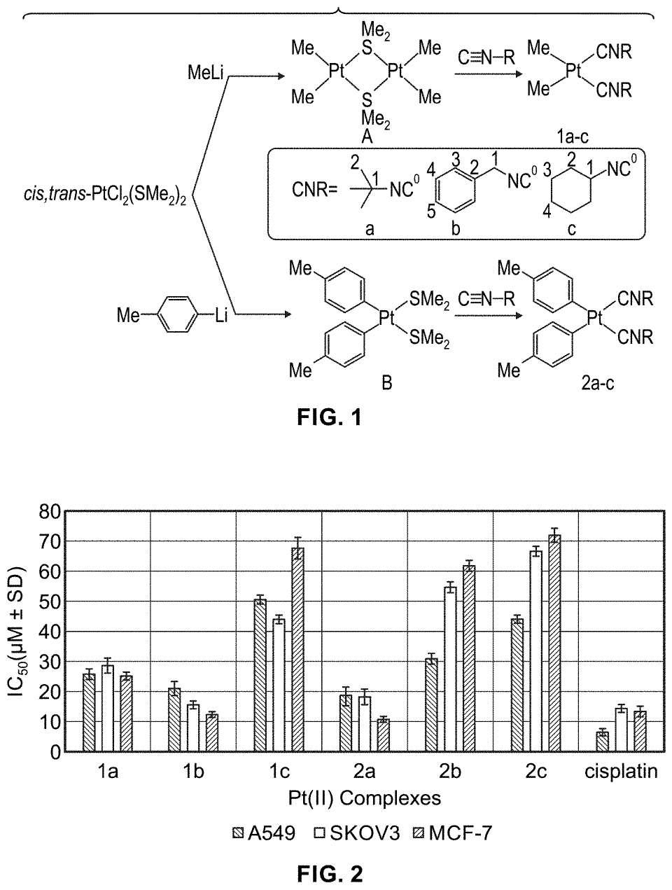

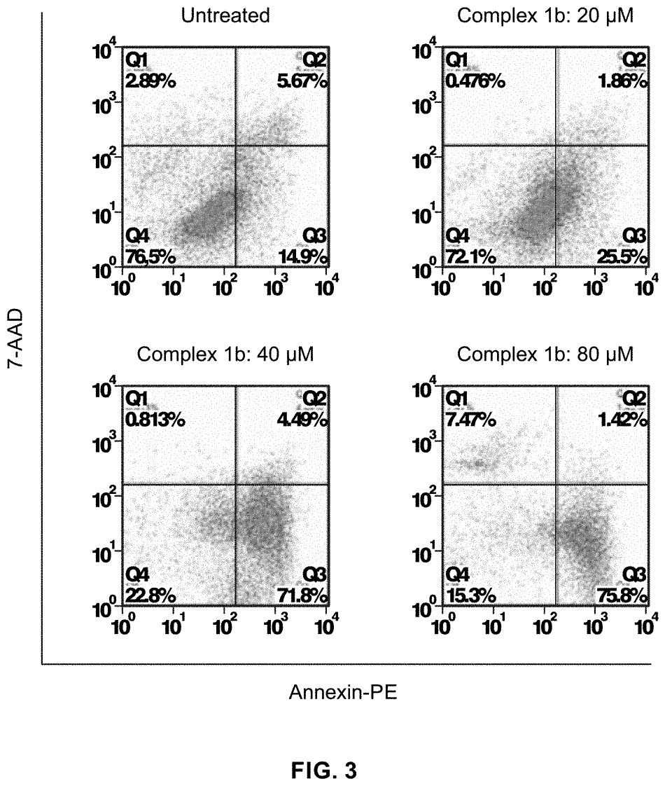

For preferred embodiments of the present invention, the general synthetic route is shown in FIG. 1. The known precursor complexes cis,cis-[PtMe₂ (μ-SMe₂)₂PtMe₂], A, and cis-[(p-tolyl)₂Pt(SMe₂)₂], B, were synthesized according to steps known to those of skill in the art. Dimethyl sulfide is a good leaving group, and it can be easily replaced with monodentate isocyanide ligands under mild conditions. Treatment of A with 4 equivalents or that of B with 2 equivalents of different types of isocyanide ligands (CNR=a; t-butyl, b; benzyl, and c; cyclohexyl isocyanide) leads to the production of new platinum(II) complexes with the general formula [R′₂Pt(CNR)₂], (1a−c; R′=Me and 2a−c; R′=p-tolyl).

Biological Activity Studies

For yet other embodiments of the present invention, the in vitro cytotoxicity effects of the 1a-c and 2a-c complexes were evaluated against a panel of three cancer cell lines (lung carcinoma (A549), ovarian carcinoma (SKOV3), and breast carcinoma (MCF-7)). As shown in Table 1, in the series of 1a-c, 1b displayed greater anti-proliferative activity than others. Compared to cisplatin, 1b exhibited higher in vitro cytotoxicity against the MCF-7 cell line, with an IC50 value of 12.51 μM, compared with that measured for cisplatin, which was 13.42 μM.

TABLE 1
Cytotoxic activity of 1a-c and 2a-c
against A549, SKOV3, and MCF-7

| | IC₅₀ (μM ± SD) | | |
|---|---|---|---|
| Name | A549 | SKOV3 | MCF-7 |
| 1a | 26.10 ± 1.42 | 28.87 ± 2.47 | 25.26 ± 1.29 |
| 1b | 21.41 ± 2.18 | 15.87 ± 1.25 | 12.51 ± 1.08 |
| 1c | 50.77 ± 1.64 | 44.65 ± 1.17 | 68.33 ± 3.21 |
| 2a | 19.18 ± 2.59 | 18.54 ± 2.38 | 11.08 ± 0.59 |
| 2b | 31.19 ± 2.27 | 55.26 ± 2.19 | 62.32 ± 1.67 |
| 2c | 44.31 ± 1.38 | 67.06 ± 1.54 | 72.35 ± 2.32 |
| Cisplatin | 6.68 ± 0.72 | 14.63 ± 1.30 | 13.42 ± 1.52 |

Complex 1b presented comparable cytotoxic activity against the SKOV3 cell line with cisplatin. In this series, 1c showed less cytotoxicity than others (FIG. 2). On the other hand, in the series 2a-c, 2a indicated higher in vitro cytotoxic activity than others. Complex 2a also revealed greater anti-proliferative activity than cisplatin against the MCF-7 cell line, and its cytotoxicity against the SKOV3 cell line was comparable with that of cisplatin. Complex 2c showed less cytotoxic activity than other complexes in this series (Table 1). Moreover, 1b and 2a displayed higher cytotoxicity against the MCF-7 cancer cell line than the cycloplatinated complexes containing 1,1′-bis(diphenylphosphino)ferrocene, tetrakis(1-3-diazinane-2-thione)platinum(II) chloride monohydrate complex, and dichlorido-platinum(II) complexes with kinetin derivatives.

Structure-activity relationship studies on these classes of compounds reveal that in the series 1a-c, which contains methyl groups bonded to platinum(II), the presence of benzyl ligand (in complex 1b) increases and the presence of cyclohexyl ligand (in complex 1c) decreases the cytotoxicity as compared to the case of other samples. In the series complexes 2a-c, due to the presence of a toluene ring bonded to platinum(II), the presence of tert-butyl ligand (in complex 2a) increases the cytotoxic activity as compared to the case of other complexes. A part of these data can be obtained from the interaction of these complexes with DNA as their target. Due to their structures that contain a phenyl ring, complexes 1b and 2a fit better in the DNA minor groove and show improved binding energies to DNA.

As shown in Table 2, in both series, an increase in the surface area (grid) and lipophilicity (logP) of the compounds decreases the cytotoxic activity.

TABLE 2
Molecular docking studies on DNA structures

Docking binding energy^{a} (kcal mol⁻¹)

| Ligand/receptor | 1BNA^{b} | 1LU5^{c} |
|---|---|---|
| 1a | −4.31 | −3.52 |
| 1b | −8.06 | −5.33 |
| 1c | −4.13 | −3.69 |
| 2a | −6.35 | −6.25 |
| 2b | −5.76 | −5.27 |
| 2c | −5.17 | −4.89 |
| Cocrystal-ligand | — | −4.78 |

^{a}All the docking protocols were performed on validated structures with RMSD values below 2 Å.
^{b}Structure of a B-DNA dodecamer.
^{c}Asymmetric platinum complex {Pt(ammine)(cyclohexylamine)}²⁺ bound to a dodecamer DNA duplex.

However, in complexes, 1a-c, the optimum log P was below 2. In complexes 2a-c with molecular weight more than 500 Da, it was observed that the more the mass, the less the cytotoxicity. On the other hand, in the complexes 1a-c, it was observed that the more the mass, the more the cytotoxicity. From the steric hindrance point of view, complexes 1c and 2c showed weak cytotoxicity in both series due to the greater steric hindrance of the cyclohexyl group.

Based on classical chemotherapy, DNA is considered to be an appropriate target for Pt-based anticancer agents. The extended π-system affects the biological behavior of the complexes and plays an essential role in their mode of actions. The lipophilicity of complexes would certainly affect the movement of complexes across the cell membranes and accordingly influence the cytotoxic activities. Steric hindrance is another event that can lead to the appearance of different antitumor behaviors of complexes as compared to that of cisplatin. However, additional interactions between the characteristics CN functional groups of Pt(II)-isocyanide complexes and N7 of guanine bases in the DNA result in different structural distortions in the DNA duplex in comparison with the case of cisplatin; this ultimately leads to cell death. It is considered that significant antiproliferative effects of complexes 1b and 2a are attributed to the strong binding interactions of the complexes to DNA. The synthesized Pt(II)-isocyanide complexes are also stable in the biological medium and may not undergo reduction or loss of ligands; on the other hand, they are insoluble in water. Moreover, the isocyanide complexes show structural stability in the presence of chloride ions.

Determination of the Apoptotic Effect of Complex 1b on the MCF-7 Cell Line

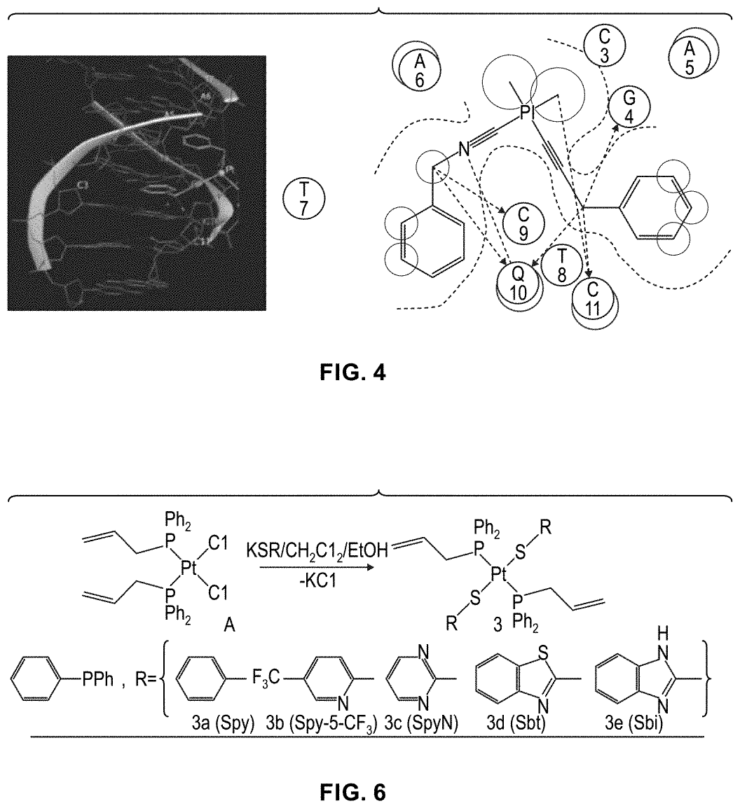

An Annexin V/7AAD kit was used to assess the ability of complex 1b, one of the most effective compounds, in apoptosis induction on the MCF-7 cell line. At the early stage of apoptosis, the symmetry of the cell membrane is lost, and phosphatidylserine (PS), which is normally found on the intracellular leaflet in live cells, is transferred to the external leaflet. Annexin V specifically recognizes and binds to PS. 7-AAD, a DNA-binding reagent, enters the nucleus of dead cells (necrotic or late apoptotic cells) and stains the DNA. As demonstrated in FIG. 4, complex 1b effectively induced apoptosis in MCF-7 cells. With an increase in the concentration, the percentages of apoptotic cells (Annexin V⁺ cells) were significantly increased from 27.36% at 20 mM to 77.22% at 80 uM. On the other hand, the number of living cells (Annexin V⁻/7-AAD⁻cells) remarkably decreased (from 72.1% to 15.3%).

Molecular Docking Analysis

The anticancer mechanism of action of platinum is likely through its intercalation with the DNA base pairs. Hence, molecular docking studies were conducted on the synthesized platinum(II) complexes to determine the specific binding site, binding mode, and the geometry of their binding to DNA.

The docking binding energies of the synthesized Pt(II) complexes with DNA (two different PDB structures of DNA) are shown in Table 2. Top-ranked binding energies (kcal mol⁻¹) in the AutoDock dlg output file were considered as a response in each run. As shown in Tables 2, complex 1b shows enhanced docking binding energies (−8.06 kcal mol⁻¹) in binding to 1BNA. In addition, complex 2a, another presented complex with high cytotoxic activity, showed stronger docking binding energies (−6.25 kcal mol⁻¹) in binding to 1LU5 as compared to other complexes. Complexes 1a and 1c showed weakest binding energies to DNA. The complexes 1b and 2a-c had better docking binding energies as compared to the cocrystal-ligand (Pt(ammine)(cyclohexylamine)²⁺) of 1LU5.

The docked model suggests that complex 1b interacts with the minor groove of DNA (FIG. 4). The methyl groups attached to platinum(II) are placed away from the base pairs in the minor groove, and the benzyl groups that are bonded to isocyanide fit into the minor groove of DNA. Complex 1b interacts via its benzyl CH₂ groups with C9, G10, G4, and C11 base pairs. The interaction of methyl group with C11 and that of isocyanide nitrogen with G10 was also observed.

Interaction of complexes 1b and 2a with DNA

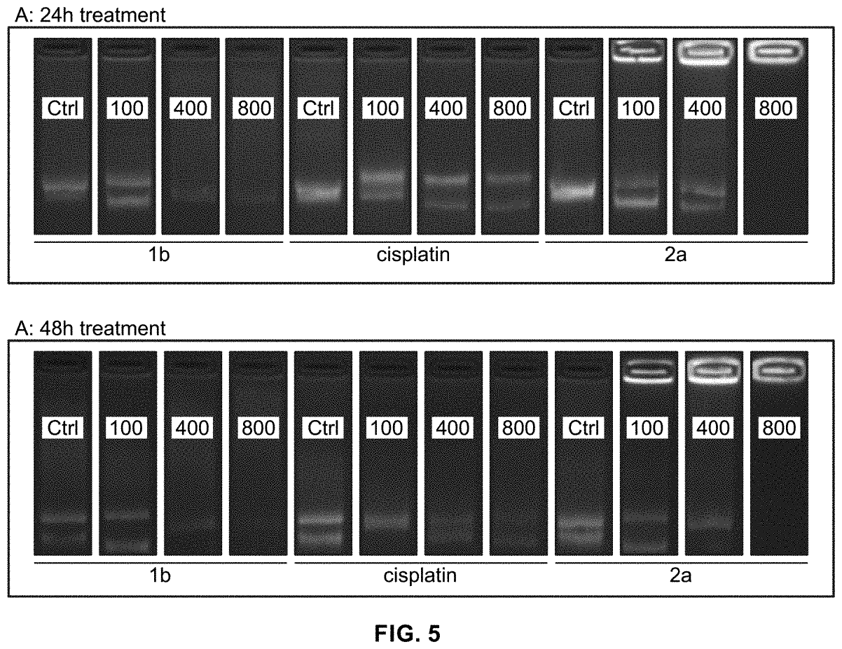

Electrophoresis mobility shift assay is a common method that is applied to show the interaction of compounds with DNA. As illustrated in FIG. 5, upon performing this assay on complexes 1b and 2c as well as cisplatin, as a positive control, it was observed that similar to cisplatin, both complexes 1b and 2a could make a nick in the DNA and provide an obvious shift in the mobility of the plasmid as compared to the case of untreated DNA. The shift was more notable with an increase in the concentrations from 100 to 800 uM, especially in the case of complex 2a; however, this compound at a higher concentration seemed to interact with the KBC loading dye and was trapped in the wells. These results collectively indicated the direct interaction of our compounds with DNA.

In other embodiments, the present invention concerns a series of closely related platinum(II) complexes with general formula trans-[Pt(PPh₂allyl)₂(κ¹—S—SR)₂], 3, PPh₂allyl=allyldiphenylphosphine, SR=deprotonated form of pyridine-2-thiol (Spy, complex 3a), 5-(trifluoromethyl)-pyridine-2-thiol (SpyCF₃-5, complex 3b), pyrimidine-2-thiol (SpyN, complex 3c), benzothiazole-2-thiol (Sbt, complex 3d) and benzimidazole-2-thiol (Sbi, complex 3e), which were synthesized by reacting a starting complex cis-[Pt(PPh₂allyl)₂Cl₂], A, with corresponding thionate ligands. The platinum complexes were spectroscopically characterized by NMR and HR ESI-Mass techniques while a single crystal X-ray crystallography demonstrated the trans-configuration of ligands around the Pt(II) center. The synthesized trans-Pt(II) thiolate complexes were used to treat three human cancer cell lines, lung (A549) ovarian (SKOV3) and breast (MCF-7) and have antitumor effects similar to cisplatin. The effects of these compounds on the proliferation of non-tumoral cell line (MCF-10A; normal human epithelial breast cell line) showed suitable selectivity among tumorigenic and non-tumorigenic cell lines. Analyses of cell cycle progression and apoptosis were conducted to screen dose/time response and to study the effects of the antiproliferative mechanism. The results have illustrated that complex 3a effectively produces cell death by apoptosis-inducing activity on MCF-7 cancer cells, in a dose-dependent manner. Furthermore, electrophoresis mobility shift assay was performed to assess the direct interaction of complex 3a, as preferred cytotoxic compound, with DNA and the strong genotoxic ability was indicated through a comet assay method. Detection of cellular reactive oxygen species (ROS) was also studied for complex 3a. Consequently, antitumor results were achieved for complex 3a as an antitumor platinum drug for use as a cancer therapy drug.

In other embodiments of the present invention, the reaction of [(Me₂S)AuCl] with an equimolar amount of benzyl isocyanide (PhCH₂NC) ligand led to the formation of complex [(PhCH₂NC)AuCl] (1). Through a salt metathesis reaction, the chloride ligand in 1 was replaced by pyrimidine-2-thiolate (SpyN⁻) to afford the complex [(PhCH₂NC) Au(κ1—S-Spy)] (2). The cytotoxic activities of 1 and 2 were evaluated against three human cancer cell lines: ovarian carcinoma (SKOV3), lung carcinoma (A549) and breast carcinoma (MCF-7). Complex 2 showed higher cytotoxicity than cisplatin against SKOV3 and MCF-7 cancer cell lines. It showed a strong anti-proliferative activity with IC50 of 7.80, 6.26 and 6.14 μM, compared with that measured for cisplatin which was 7.62, 12.36 and 11.47 μM, against A549, SKOV3 and MCF-7 cell lines, respectively. The induction of cellular apoptosis by 2 was also studied on MCF-7 cell line. The results indicated that 2 induces apoptosis in cancerous cells in a dose-dependent manner.

In other embodiments of the present invention, a synthetic approach has been introduced to obtain a class of trans platinum complexes. For these embodiments as shown in FIG. 6, complexes closely related platinum(II) complexes with general formula trans-[Pt(PPh₂allyl)₂(κ¹—S—SR)₂], 3, PPh₂allyl=allyldiphenylphosphine, SR=deprotonated form of pyridine-2-thiol (Spy, complex 3a), 5-(trifluoromethyl)-pyridine-2-thiol (SpyCF₃-5, complex 3b), pyrimidine-2-thiol (SpyN, complex 3c), benzothiazole-2-thiol (Sbt, complex 3d) and benzimidazole-2-thiol (Sbi, complex 3e), were synthesized by reacting a starting complex cis-[Pt(PPh₂allyl)₂Cl₂], A, with corresponding thionate ligands. The platinum complexes were spectroscopically characterized by NMR and HR ESI-Mass techniques while a single crystal X-ray crystallography demonstrated the trans-configuration of ligands around the Pt(II) center. The synthesized trans-Pt(II) thiolate complexes are used to treat three human cancer cell lines, lung (A549) ovarian (SKOV3) and breast (MCF-7) and have shown the promising antitumor effects of complex 3a in comparison with standard cisplatin. The effects of these compounds on the proliferation of non-tumoral cell line (MCF-10A; normal human epithelial breast cell line) showed suitable selectivity among tumorigenic and non-tumorigenic cell lines. Analyses of cell cycle progression and apoptosis were conducted to screen dose/time response and to study the effects of the antiproliferative mechanism. The results have illustrated that complex 3a effectively produces cell death by apoptosis-inducing activity on MCF-7 cancer cells, in a dose-dependent manner. Furthermore, electrophoresis mobility shift assay was performed to assess the direct interaction of complex 3a, as the best cytotoxic compound, with DNA and the strong genotoxic ability was indicated through a comet assay method. Detection of cellular reactive oxygen species (ROS) was also studied for complex 3a. Consequently, complex 3a may be used as a cancer therapy drug.

For these embodiments, multinuclear ¹H, ^{31p}{¹H} and ¹⁹⁵Pt{¹H} NMR spectroscopy was applied to accurately characterize the platinum (II) compounds. Single crystal X-ray crystallography technique, confirmed the cis-configuration of PPh₂allyl ligands in the starting complex. The X-ray crystal structure determined that the heterocyclic thionate ligands are bound to the Pt(II) center with S-coordinating mode while trans-positioned to each other. Meanwhile, all platinum (II) complexes were tested against three human cancer cell lines including lung (A549), ovarian (SKOV3), and breast (MCF-7) which showed antitumor activities.

In vitro studies introduced complex 3a as a therapeutic agent due to the inhibition growth of MCF-7 cancer cell, mediated through inducing apoptosis. Furthermore, the effect of complex 3a on the MCF-7 cell was tested by cell cycle analysis to assess the mechanisms of antiproliferative effects. To predict the genotoxic effect of complex 3a on cancerous cells, a comet assay was used to show that complex 3a targets the genome content of MCF-7 cancerous cells and directly interact with DNA as its major target. Accordingly complex 3a has a strong affinity to the DNA in vitro making it useful as an antitumor agent.

Biological Activity Studies

For other preferred embodiments oft he present invention, the in vitro cytotoxic activity of A and complexes 3a-d were also evaluated on three cancer cell lines including human lung (A549), ovarian (SKOV3), and breast (MCF-7) carcinoma. As shown in Table 3, complex 3a, showed higher anti-proliferative activity than cisplatin on the studied cell lines.

TABLE 3
In vitro cytotoxicity of all the synthesized compounds
against cancerous and non-cancerous cell lines.

| | (IC₅₀ ± SD) μM | | | |
|---|---|---|---|---|
| Complex | A549 | SKOV3 | MCF-7 | MCF-10A |
| A | 22.49 ± 1.62 | 34.50 ± 1.53 | 19.01 ± 1.24 | 68.74 ± 1.21 |
| 3a | 4.31 ± 0.72 | 6.23 ± 0.74 | 4.80 ± 0.71 | 38.42 ± 2.06 |
| 3b | 12.59 ± 1.09 | 17.49 ± 1.21 | 19.68 ± 1.59 | 53.26 ± 1.13 |
| 3c | 16.01 ± 1.12 | 12.38 ± 1.17 | 15.64 ± 1.37 | 44.71 ± 1.42 |
| 3d | 20.15 ± 1.47 | 22.14 ± 1.67 | 34.65 ± 0.83 | 88.42 ± 1.39 |
| 3e | 18.62 ± 1.55 | 21.73 ± 1.19 | 17.45 ± 1.71 | 47.06 ± 0.83 |
| Cisplatin | 9.71 ± 1.70 | 14.48 ± 1.54 | 11.59 ± 1.66 | 29.47 ± 1.03 |

The complex showed a good anti-proliferative activity with IC₅₀ of 4.31, 6.23 and 4.80 μM comparing with those measured for cisplatin (9.71, 14.48 μM and 11.59 μM, against A549, SKOV3 and MCF-7 cell lines, respectively). A one-way ANOVA statistical analysis showed that the differences between 3a and cisplatin is statistically significant. 3b also displayed better in vitro cytotoxicity than cisplatin on SKOV3 cell line with IC₅₀ of 12.38 μM, however this difference is not statistically significant. Complex 3b and cisplatin IC₅₀ on A549 cell lines was also not statistically significant. Complex 3c and cisplatin IC₅₀ on SKOV3 cells was also not statistically significant. Complexes A, 3b, 3c, and 3e also showed antitumor activitiy against MCF-7 cell line in comparison with cisplatin. It should be mentioned that, the cytotoxicity of all the ligands including Spy, Spy-5-CF₃, SpyN, Sbt, Sbi and allydiphenylphosphane (PPh₂allyl) was evaluated against A549 cell line and the IC50 of all of these ligands was more than 100 μM.

In addition, to verify the selectivity between cancer and normal cell line, the effects of these compounds on the proliferation of non-tumoral cell line (MCF-10A; normal human epithelial breast cell line) were also determined. As shown in Table 3, all of these compounds displayed reasonable selectivity between tumorigenic and non-tumorigenic cell lines and showed less cytotoxicity than cisplatin on MCF-10A cell line. Structure-activity relationship studies revealed that, generally, the complex A which encompasses chloro group, instead of thiolated ligand, showed lower anti-proliferative activity than others. Among the thiolated ligands, complex 3b with a thiolated pyrimidine ring and especially, complex 3a with a thiolated pyridine ring showed the highest potency. In order to gain some more insight in the structure-activity relationship of the synthesized trans-Pt(II) complexes, the chemical descriptors of them such as surface area, volume, hydration energy, logP (measure of lipophilicity), refractivity (measuring the total polarizability of a mole of a compound), polarizability and mass were calculated using Hyperchem 8 software. The greater logP (lipophilicity) of complex 3a confirmed the importance of this descriptors for the cytotoxic activity of the trans-Pt(II) complexes.

Determining Apoptotic Effect of Complex 3a on MCF-7 Cell Line

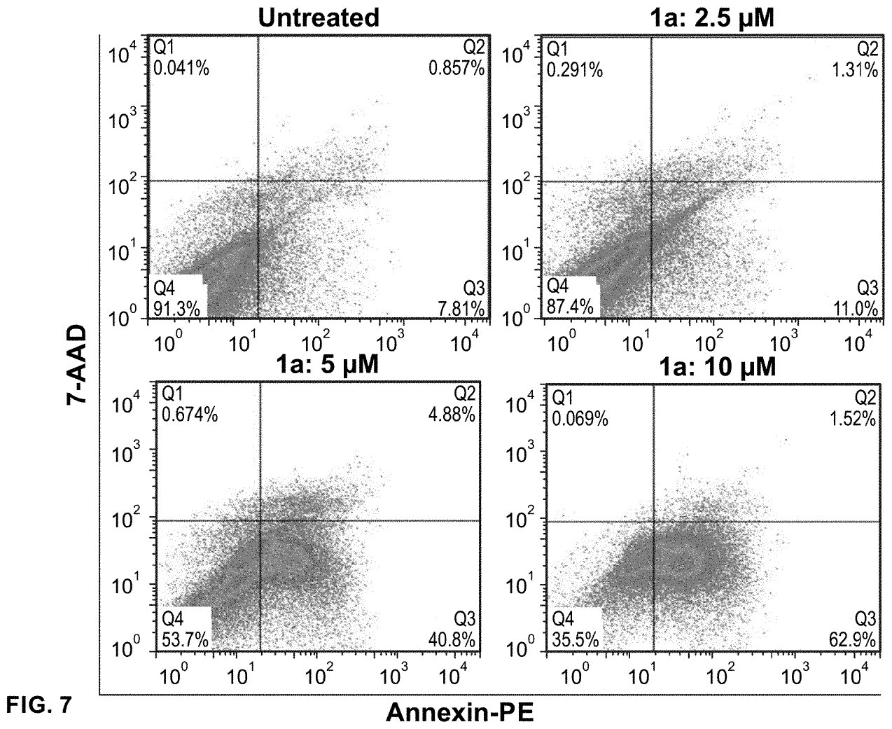

BioLegend's PE Annexin V Apoptosis Detection Kit was used with 7AAD to specifically determine the dose-dependent apoptotic effect of complex 3a on cancerous cells. To determine this, complex 3a with three concentrations (2.5, 5 and 10 μM) was applied onto MCF-7 cells. As illustrated in FIG. 7, with the increase in the concentration of complex 3a from 2.5 to 10 μM, the percentage of the cells in early apoptotic phase significantly increases from 7.8% in untreated cells to 11.0%, 40.8%, and 62.9% in the treated cells. This observation indicated that complex 3a as a representative of new trans-Pt (II) series, is able to effectively induce apoptosis in cancerous cells in a dose dependent manner. Also, the observed antiproliferative/cytotoxic effect for complex 3a in cytotoxic assay, could be partly mediated through inducing apoptosis in cancer cells.

The Potential Effect of Complex 3a on the MCF-7 Cells' Cell Cycle

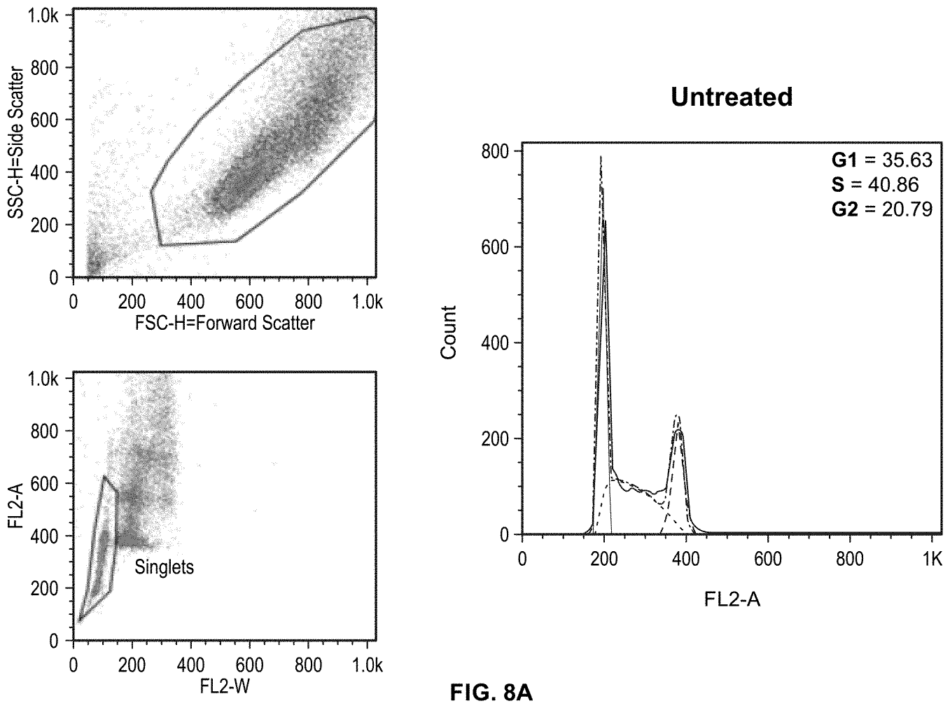

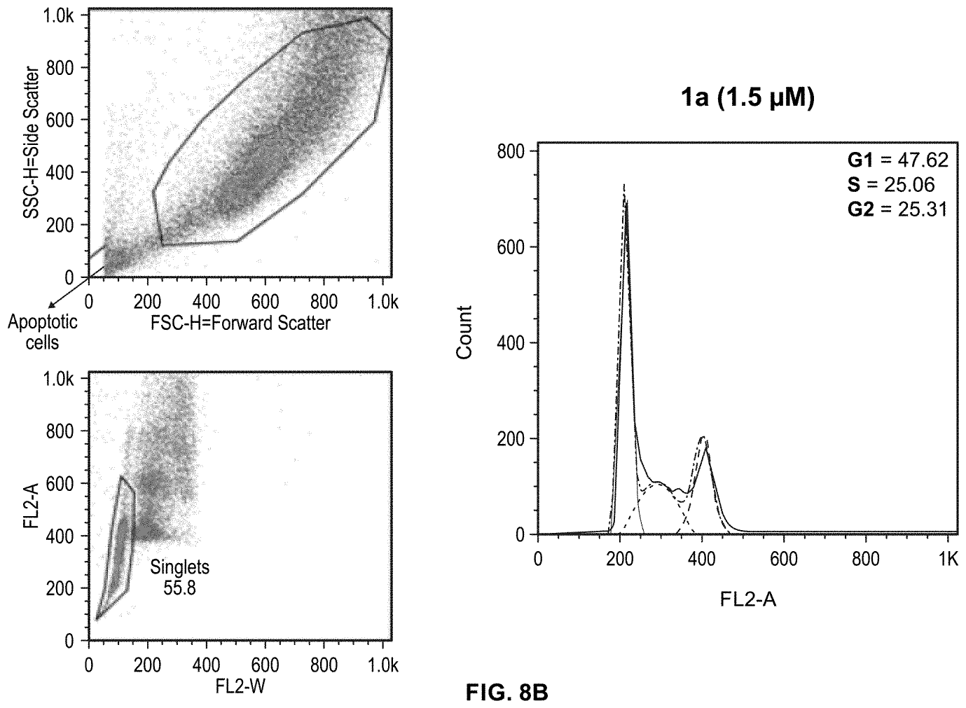

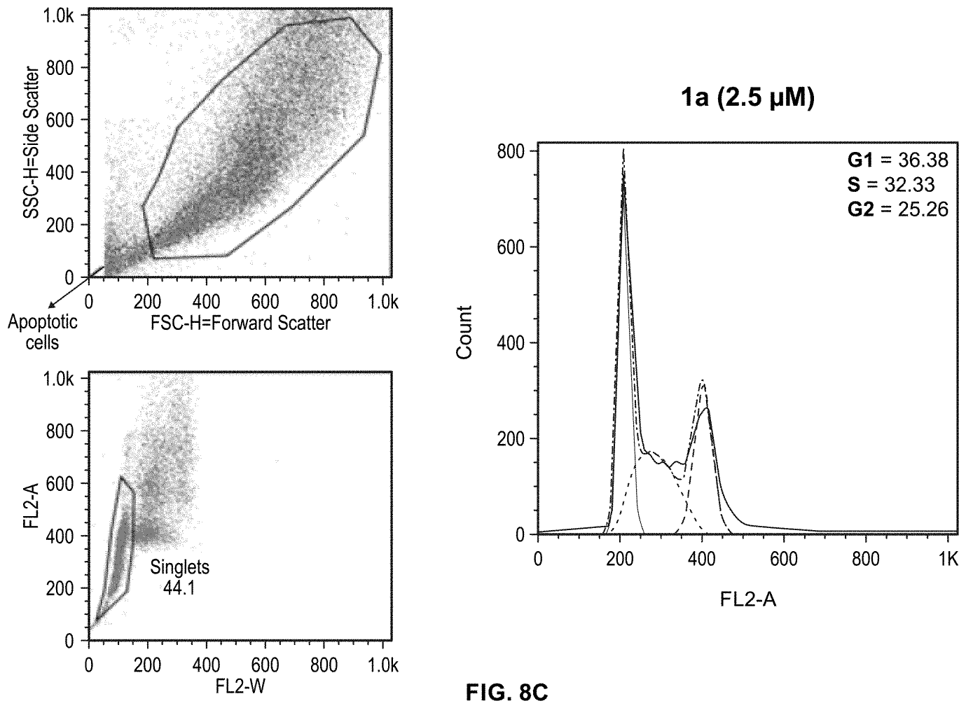

Quantitation of DNA content using flow cytometry or cell cycle analysis, is a basic method which is commonly used to assess the mechanisms of antiproliferative effects of anticancer drugs. In this method, a fluorescent DNA binding dye (in the present study: propidium iodide, PI) is used to stain DNA and measure the amount of DNA present in the cell. The cells in the G2 phase are expected to absorb approximately twice the amount of color compared to the cells in G1 as their DNA content has been doubled during S phase. Therefore, the cells in S phase have more DNA than G1 cells but less than G2 ones. Hence, one can check whether the embodiments of the present compound exerts antitumor effects thorough modifying cell cycle or not. As could be observed in FIG. 8, comparing to untreated cells, no obvious change in different cycle phases could be observed which probably shows that complex 3a has no clear effect on the cell cycle of cancerous cells.

Genotoxicity and DNA Interaction

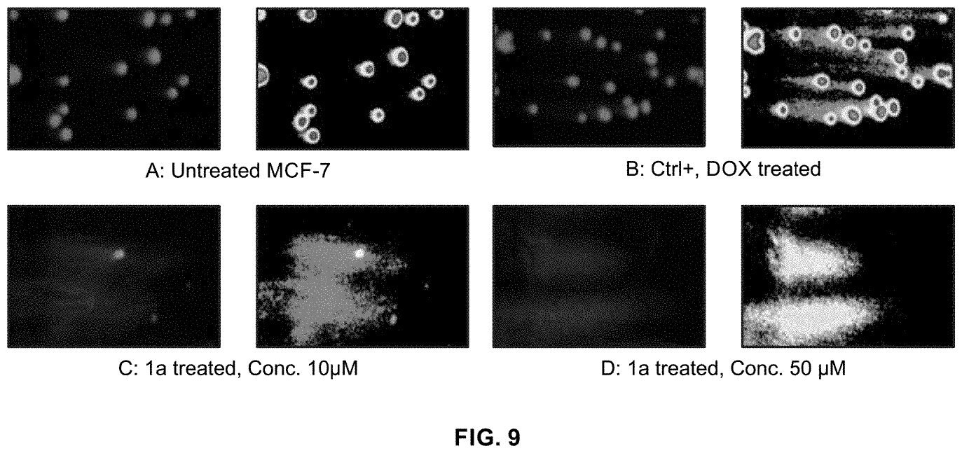

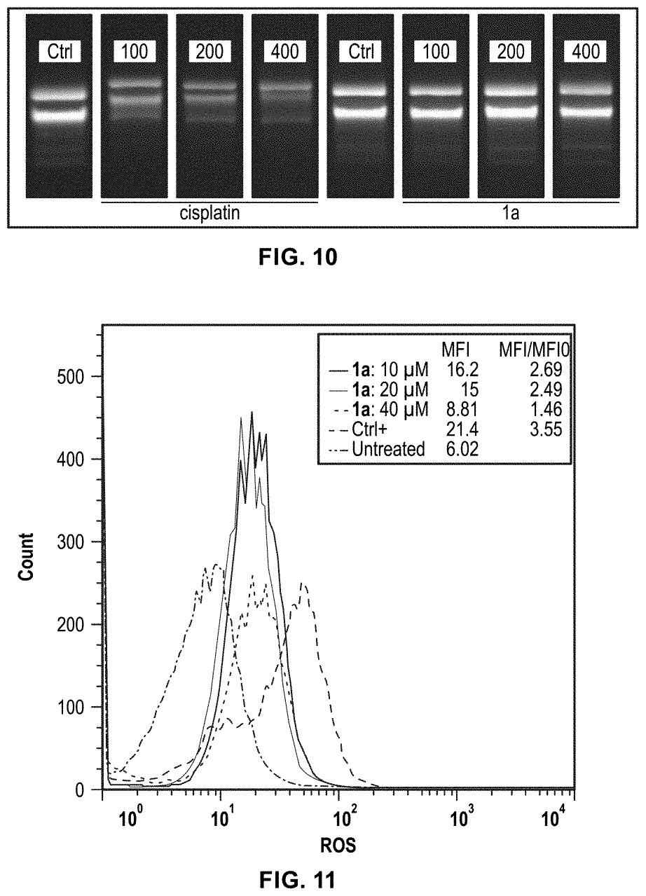

Here, comet assay was used as a valuable method to predict the genotoxic effect of new synthesized trans-Pt(II) complexes on cancerous cells. In this single cell microgel electrophoresis method, following the DNA damage, the migration of chromosomal DNA from the nucleus increases and resembles the shape of a tail or comet. The longer tails display the more genotoxicity, while untreated cells as un-fragmented cells, represent a little or no tail. The genotoxicity of complex 3a was found to be the best cytotoxic complex in the 1 series through comet assay. As could be observed in FIG. 9, treatment of MCF-7 cells with both low and high concentrations of complex 3a (10 and 50 μM), results in the appearance a relatively long tail following the electrophoresed cells in the concentration of 10 μM, which shows strong genotoxic ability of complex 3a. In the case of a concentration of 50, as displayed in FIG. 9D, no nucleus remained and only a blurry tail of degraded DNA could be seen. These observations collectively showed that complex 3a intensely targets the genome content of cancerous cells. However, in electrophoresis mobility shift assay which used to further show the direct interaction of complex 3a with DNA, a little shift was observed comparing to cisplatin, as positive control (FIG. 10). It could also be seen that compared to the untreated control, complex 3a could make a nick in DNA and subsequently slightly shift the mobility of the plasmid in a dose dependent manner. These observations confirm the direct interaction of complex 3a with DNA through genotoxic effect as observed in comet assay. This clearly indicates that the effective mechanisms of these compounds have a direct interaction with DNA and probably other molecules as well. These results are collectively consistent with previous studies which have described platinum compounds as DNA-targeting metal-based anticancer agents.

In order to determine the binding mode and binding site of the trans-Pt(II) complexes in interaction with DNA, molecular docking studies was also employed. The high negative values of the binding free energies (kcal/mol) of the trans-Pt(II) complexes suggest that they bind reasonably well to DNA. Complex 3d showed the lowest binding energies in compared to the others. As a result, complex 3d was determined to have fitted in to the minor groove of DNA and interacts through its sulfur groups via weak hydrogen bonding with G4 and C11 base pairs in minor groove of DNA.

The percentage of degraded DNA in the tail has remarkably increased following treatment with doxorubicin (Dox) as positive control. This is also effected by differences in the concentrations of complex 3a in comparison to untreated cells (negative control). pGEM-FT plasmid in circular form was incubated with different concentrations of cisplatin (positive controls) as well as complex 3a for 24 h.

Intracellular Reactive Oxygen Species (ROS) Determination

Flow cytometry was used for determination of cellular reactive oxygen species (ROS) in SKOV3 cell line after treatment with complex 3a. In this highly sensitive method, to consider ROS formation, SKOV3 cells were treated with complex 3a and H₂O₂ (positive control) and stained with 2′,7′-Dichlorodihydrofluorescein diacetate (DCFH-DA), a cell-permanent non-fluorescent dye which is oxidized by cellular ROS and produce 2′,7′-dichlorofluorescein (DCF) fluorescent component. The intensity of DCF is a direct estimate of the amount of ROS within the cells. As illustrated in the FIG. 11, treatment of SKOV3 cells with complex 3a moderately induce ROS in a relatively dose dependent manner, however the production of ROS was higher in lower concentration of complex 3a (10 μM) comparing to higher concentration (40 μM) (FIG. 11).

While the foregoing written description enables one of the ordinary skill in the art to make and use what is considered presently to be the best mode thereof, the skilled artisan will understand and appreciate the existence of variations, combinations, and equivalents of the specific embodiment, method, and examples herein. The disclosure should therefore not be limited by the above described embodiments, methods, and examples, but by all embodiments and methods within the scope and spirit of the disclosure.

What is claimed is:

1. A method of treating cancer which comprises administering to a cancer patient a therapeutically effective amount of one or more metal precursor complexes with one or more ligands.

2. The method of claim 1 wherein said metal precursor is cis;cis-[PtMe₂(μ-SMe₂)₂PtMe₂].

3. The method of claim 2 wherein said ligand is an isocyanide, said isocyanide ligand is [Me₂Pt(CNR)₂](R=t-butyl).

4. The method of claim 2 wherein said ligand is an isocyanide, said isocyanide ligand is [Me₂Pt(CNR)₂] (R=benzyl).

5. The method of claim 2 wherein said ligand is an isocyanide, said isocyanide ligand is [Me₂Pt(CNR)₂] (R=cyclohexyl).

6. The method of claim 1 wherein said metal precursor is cis-[(p-tolyl)₂Pt(SMe₂)₂].

7. The method of claim 2 wherein said ligand is an isocyanide, said isocyanide ligand is [(p-tolyl)₂Pt(CNR)₂](R=t-butyl).

8. The method of claim 2 wherein said ligand is an isocyanide, said isocyanide ligand is [(p-tolyl)₂(CNR)₂] (R=benzyl).

9. The method of claim 2 wherein said ligand is an isocyanide, said isocyanide ligand is [(p-tolyl)₂(CNR)₂] (R=cyclohexyl).

10. The method of claim 2 wherein said isocyanide ligand is [Me₂Pt(CNR)₂] (R=benzyl) and said complex binds to a DNA minor grove.

11. The method of claim 10 wherein methyl groups attach to said metal precursor and are placed away from the base pairs in the DNA minor groove.

12. The method of claim 11 wherein said benzyl groups bonded to said isocyanide fit into the minor groove of DNA.

13. The method of claim 12 wherein said complex interacts via benzyl CH₂ groups with C9, G10, G4, and C11 base pairs of the DNA.

14. The method of claim 1 wherein said metal precursor is [(Me₂S)AuCl] and said ligand, said ligand is a benzyl isocyanide (PhCH₂NC) and forms complex [(PhCH₂NC)AuCl].

15. The method of claim 14 wherein said chloride ligand is replaced by pyrimidine-2-thiolate (SpyN⁻) to form complex [(PhCH2NC) Au(κ¹—S-Spy).

16. The method of claim 1 wherein said metal precursor is [Pt(PPh₂allyl)₂Cl₂] and said ligand is a thionate ligand.

17. A method of treating cancer which comprises administering to a cancer patient a therapeutically effective amount of platinum complex having a formula trans-[Pt(PPh₂allyl)₂(κ¹—S—SR)₂], PPh₂allyl=allyldiphenylphosphine and SR is a deprotonated form of pyridine-2-thiol.

18. The method of claim 17 wherein is SR from the group comprising 5-(trifluoromethyl)-pyridine-2-thiol; pyrimidine-2-thiol; benzothiazole-2-thiol; or benzimidazole-2-thiol.

19. A method of treating cancer which comprises administering to a cancer patient a therapeutically effective amount of a formula having Pt(II) complexes synthesized by the reaction of the precursor complexes cis,cis-[Me₂Pt(μ-SMe₂)₂PtMe₂], and cis-[(p-tolyl)₂Pt(SMe₂)₂], with four and two equivalents of different types of isocyanide ligands (CNR; R=a; t-butyl, b; benzyl, and c; cyclohexyl isocyanide), respectively.
